(19) United States
(12) Patent Application Publication
(10) Pub. No.: US 2025/0107835 A1
(43) Pub. Date: Apr. 3, 2025

(54) CATHETER WITH MAPPING STRUCTURE ABOUT ABLATION ELECTRODE

(71) Applicant: Boston Scientific Scimed, Inc., Maple Grove, MN (US)

(72) Inventors: Eric Buck, Shoreview, MN (US); Brendan Early KOOP, Ham Lake, MN (US)

(21) Appl. No.: 18/895,137

(22) Filed: Sep. 24, 2024

Related U.S. Application Data
(60) Provisional application No. 63/586,903, filed on Sep. 29, 2023.

(51) Int. Cl. A61B 18/00

(52) U.S. Cl. CPC A61B 18/00; A61B 2018/00077; A61B 2018/00184; A61B 2018/00351; A61B 2018/00577; A61B 2018/00613; A61B 2018/00839

(57) ABSTRACT

A catheter for ablation of tissue through irreversible electroporation is disclosed. The catheter includes a tubular outer shaft having a distal end, a mapping electrode assembly extending distally from the distal end of the outer shaft, and an ablation electrode assembly. the electrode assembly defines a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes; and an ablation electrode assembly extending distally from the distal end of the outer shaft, the ablation electrode assembly disposed in the inner space.

### CROSS REFERENCE TO RELATED APPLICATIONS

The present application claims priority to U.S. Provisional Patent Application No. 63/586,903, filed Sep. 29, 2023, the entire disclosure of which is incorporated herein in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a catheter for ablating cardiac tissue through irreversible electroporation, the catheter comprising a tubular outer shaft having a distal end; a mapping electrode assembly extending distally from the distal end of the outer shaft, the mapping electrode assembly defining a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes; and an ablation electrode assembly extending distally from the distal end of the outer shaft, the ablation electrode assembly disposed in the inner space.

In Example 2, the catheter of Example 1, wherein the ablation electrode assembly is movable with relation to the mapping electrode assembly in the expanded configuration.

In Example 3, the catheter of any of Examples 1-2, further comprising a hub sensing electrode centrally located on the central hub portion of the electrode assembly.

In Example 4, the catheter of any of Examples 1-3, wherein the splines each have lateral edges having an atraumatic shape.

In Example 5, the catheter of any of Example 1-4, wherein the ablation electrode assembly includes a central post and an ablation electrode, the central post extending distally from the distal end of the tubular shaft and into the inner space defined by the mapping electrode assembly when the mapping electrode assembly is in an expanded configuration, wherein the ablation electrode is disposed on the central post.

In Example 6, the catheter of any of Examples 1-5, wherein the ablation electrode assembly includes a circular surface of an exposed conductive material generally perpendicular to a longitudinal axis of the catheter.

In Example 7, the catheter of any of Examples 1-5, wherein the ablation electrode assembly includes a domed shape of an exposed conductive material.

In Example 8, the catheter of any of Examples 1-5, wherein the ablation electrode assembly includes a bulbous shape of an exposed conductive material.

In Example 9, the catheter of any of Examples 1-8, wherein the ablation electrode assembly includes a plurality of ablation electrodes.

In Example 10, the catheter of any of Examples 1-9, wherein a movable spine of the plurality of splines is angularly movable with respect to another spline of the plurality of splines.

In Example 11, the catheter of any of Examples 1-9, wherein each spline of the plurality of splines are fixed in relation to all other splines of the plurality of splines.

In Example 12, the catheter of any of Examples 1-11, wherein the plurality of splines includes a support member.

In Example 13, the catheter of any of Examples 1-11, wherein each of the splines includes a flex circuit comprising the spline sensing electrodes.

In Example 14, the catheter of Example 13, wherein each of the splines further includes a support member having an outer surface and the flex circuit comprising the spline sensing electrode is disposed on the outer surface of the support member.

In Example 15, the catheter of Example 13, wherein the support member comprises a nickel-titanium alloy.

In Example 16, a catheter for ablating cardiac tissue through irreversible electroporation, the catheter comprising a tubular outer shaft having a distal end; a mapping electrode assembly extending distally from the distal end of the outer shaft, the mapping electrode assembly defining a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes; and an ablation electrode assembly extending distally from the distal end of the outer shaft, the ablation electrode assembly disposed in the inner space.

In Example 17, the catheter of Example 16, wherein the ablation electrode assembly is movable with relation to the mapping electrode assembly in the expanded configuration.

In Example 18, catheter of Example 16, further comprising a hub sensing electrode centrally located on the central hub portion of the electrode assembly.

In Example 19, the catheter of claim 16, wherein the splines each have lateral edges having an atraumatic shape.

In Example 20, the catheter of Example 16, wherein the ablation electrode assembly includes a central post and an ablation electrode, the central post extending distally from the distal end of the tubular shaft and into the inner space defined by the mapping electrode assembly when the mapping electrode assembly is in an expanded configuration, wherein the ablation electrode is disposed on the central post.

In Example 21, the catheter of Example 16, wherein the ablation electrode assembly includes a circular surface of an exposed conductive material generally perpendicular to a longitudinal axis of the catheter.

In Example 22, the catheter of Example 16, wherein the ablation electrode assembly includes a domed shape of an exposed conductive material.

In Example 23, the catheter of Example 16, wherein the ablation electrode assembly includes a bulbous shape of an exposed conductive material.

In Example 24, the catheter of Example 16, wherein the ablation electrode assembly includes a plurality of ablation electrodes.

In Example 25, the catheter of Example 16, wherein a movable spine of the plurality of splines is angularly movable with respect to another spline of the plurality of splines.

In Example 26, the catheter of Example 16, wherein each spline of the plurality of splines are fixed in relation to all other splines of the plurality of splines.

In Example 27, the catheter of Example 16, wherein the plurality of splines includes a support member.

In Example 28, the catheter of Example 16, wherein each of the splines includes a flex circuit comprising the spline sensing electrodes.

In Example 29, the catheter of Example 28, wherein each of the splines further includes a support member having an outer surface and the flex circuit comprising the spline sensing electrode is disposed on the outer surface of the support member.

In Example 30, the catheter of Example 28, wherein the support member comprises a nickel-titanium alloy.

In Example 31, a catheter for ablating cardiac tissue through irreversible electroporation, the catheter comprising a tubular outer shaft having a distal end; a mapping electrode assembly extending distally from the distal end of the outer shaft, the electrode assembly defining a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes disposed on an outer surface of a support member; and an ablation electrode assembly extending distally from the distal end of the outer shaft, wherein the ablation electrode assembly includes a central post and an ablation electrode, the central post extending distally from the distal end of the tubular shaft and into the inner space defined by the mapping electrode assembly when the mapping electrode assembly is in an expanded configuration, wherein the ablation electrode is disposed on the central post.

In Example 32, the catheter of Example 31, wherein each of the splines includes a flex circuit comprising the spline sensing electrodes.

In Example 33, the catheter of Example 31, wherein the ablation electrode includes an exposed conductive material generally perpendicular to a longitudinal axis of the catheter.

In Example 34, a catheter for ablating cardiac tissue through irreversible electroporation, the catheter comprising a tubular outer shaft having a distal end; a mapping electrode assembly extending distally from the distal end of the outer shaft, the mapping electrode assembly defining a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes, wherein a movable spine of the plurality of splines is angularly movable with respect to another spline of the plurality of splines; and an ablation electrode assembly extending distally from the distal end of the outer shaft, the ablation electrode assembly disposed in the inner space, wherein the ablation electrode assembly is movable with relation to the mapping electrode assembly in the expanded configuration.

In Example 35, the catheter of Example 34, wherein the ablation electrode assembly includes a central post and an ablation electrode, the central post extending distally from the distal end of the tubular shaft and into the inner space defined by the mapping electrode assembly when the mapping electrode assembly is in an expanded configuration, wherein the ablation electrode is disposed on the central post.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2A is a perspective illustration of a distal portion of a splined catheter for use in the electrophysiology system of FIG. 1, in accordance with embodiments of the subject matter of the disclosure.

FIGS. 2B-2D are perspective illustrations of a distal portion of alternative embodiments of the spline catheter of FIG. 2A.

FIG. 3 is a perspective illustration of another distal portion of a splined catheter for use in the electrophysiology system of FIG. 1, in accordance with embodiments of the subject matter of the disclosure.

FIGS. 4A-4C are illustrations of the distal portion of the catheter of FIG. 1 in exemplary use settings within a cardiac chamber of a patient, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

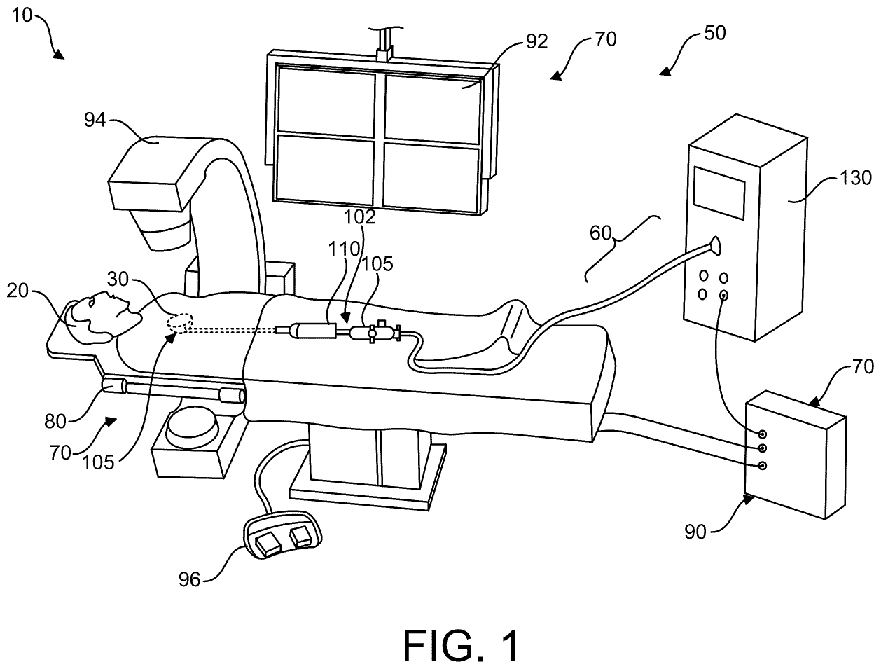

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation catheter system 60 includes an electroporation catheter 100 having a proximal portion 102 and a distal portion 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the electroporation catheter 100, in particular all or part of the distal portion 105 thereof, can be deployed to the specific target sites within the patient's heart 30.

In embodiments, the electroporation catheter system 60 is configured to deliver electric field energy to targeted tissue in the patient's heart 30 to create tissue apoptosis, rendering the tissue incapable of conducting electrical signals.

The electroporation console 130 is configured to control functional aspects of the electroporation catheter system 60. In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the electroporation catheter 100 in a monopolar or bipolar mode of operation, as will be described in further detail herein.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

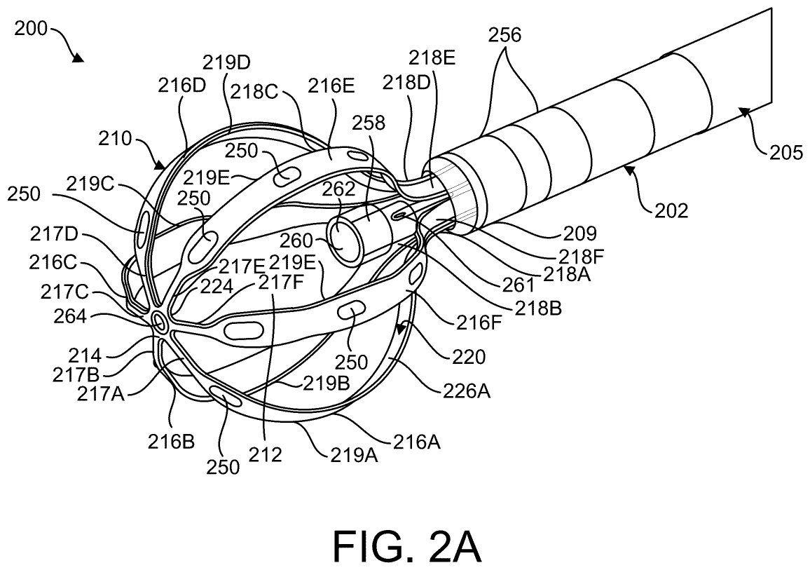

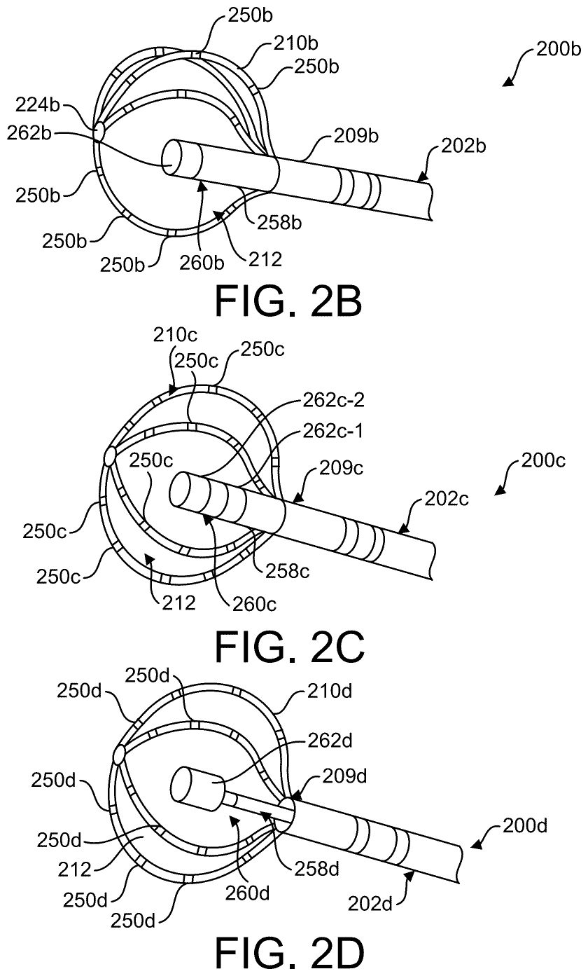

FIG. 2A is a partial perspective illustration of an electroporation catheter 200 having a catheter distal portion 205 according to an embodiment of the present disclosure. The electroporation catheter 200 corresponds to the electroporation catheter 100 described with respect to FIG. 1. The electroporation catheter 200 has a tubular outer shaft 202 having a shaft distal end 209, a mapping electrode assembly 210 extending distally from the distal end 209 of the outer shaft 202. The mapping electrode assembly 210 in embodiments is coupled to the distal end 209 either directly or indirectly such as via a coupling mechanism that may include a hinge. In embodiments, the mapping electrode assembly 210 is configured to expand from a collapsed configuration when constrained within a delivery sheath to a pre-defined expanded configuration defining an inner space 212. The electroporation catheter 200 also includes an ablation electrode assembly 260 extending distally from the distal end 209 of the outer shaft 202 and disposed in the inner space 212. The ablation electrode 260 is configured to receive pulsed electrical signals/waveforms from the electroporation console 130 (FIG. 1), thereby creating pulsed electric fields sufficient for ablating target tissue via irreversible electroporation. Additionally, the mapping electrode assembly 210 further includes a plurality of mapping and sensing electrodes 250 configured for sensing cardiac electrical signals, localization of the electrode assembly 210 within the patient anatomy (e.g., via the EAM system 70 of FIG. 1), and determining proximity to target tissue within the anatomy.

Overall, the ablation electrode assembly 260 embodiments described herein within the scope of the present disclosure, is primarily designed for the creation of relatively localized ablation lesions (i.e., focal lesions), as compared to relatively large diameter circumferential lesions created in pulmonary vein isolation procedures). However, the skilled artisan will appreciate that the teachings of the present disclosure can be readily adapted for a catheter capable of large diameter circumferential lesions. The designs of the various electrode assembly embodiments, such as mapping electrode assembly 210 and ablation electrode assembly 260, described herein can provide the clinician with a wide range of capabilities for monopolar and bipolar focal pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

The mapping electrode assembly 210 defines the inner space 212 and the ablation electrode assembly 260 is disposed within the inner space 212. In some embodiments, the ablation electrode assembly is configured to be fully disposed within an undeformed, expanded mapping electrode assembly 210. In the illustrated examples, the mapping electrode assembly 210 forms a cage about the ablation electrode assembly 260. The disclosure proceeds with the mapping electrode assembly configured as a basket having a plurality of splines or struts. In the basket configuration, the splines allow for the mapping electrode to deform when pressed against tissue and, in some embodiments, allow for the ablation electrode to extend through the mapping electrode assembly and contact the tissue. Other examples of mapping electrode assembly configurations are contemplated, such as globe or mesh electrode assemblies formed of interconnected splines or struts. In such examples, the ablation electrode assembly may or may not extend through a deformed mapping electrode assembly to contact tissue.

The mapping electrode assembly 210 as a whole has a distally-located central hub portion 214 and a plurality of splines 216A-216F extending proximally from the central hub portion 214. As further shown, each respective spline 216A-216F has a distal end portion 217A-217F, a proximal end portion 218A-218F, and an intermediate portion 219A-219F extending between the distal end portion 217A-217F and the proximal end portion 218A-218F. As shown, each of the proximal end portions 218A-218F is attached to and constrained by the distal end 209 of the outer shaft 202. As further shown, in the illustrated embodiment, the intermediate portion 219A-219F of each spline 216A-216F has a lateral width that is greater than the lateral width of each of the respective proximal end portion 218A-218F and the distal end portions 217A-217F. In embodiments, the particular geometry of the splines 216A-216F and the related components, e.g., mapping electrodes, is optimized to provide desired mechanical and therapeutic/diagnostic capabilities. In embodiments, the mapping electrode assembly 210 is configured to self-expand from a collapsed configuration when constrained within a delivery sheath to the pre-defined expanded configuration defining the inner space 212. For example, the electrode assembly 210 can be constructed from a material having shape-memory properties. In other examples, the controls on the catheter 200, such as on a proximal handle, can mechanically expand and collapse the mapping electrode assembly 210.

In the illustrated embodiment, the splines 216A-216F are composed of a support member 220 and a flexible circuit 222 secured to and disposed over an outer surface of the support member 222. In some embodiments, the splines 216A-216F do not include a flex circuit. In such embodiments, the sensing electrodes and electrical leads are coupled directly to the support member 222. The support member 220 functions, among other things, as a primary structural support of the electrode assembly 210, and thus primarily defines the mechanical characteristics of the electrode assembly 210. In embodiments, the support member 220 is formed from a superelastic material (metal or polymer) to provide desired mechanical/structural properties to the electrode assembly 210. In embodiments, the support member 220 is formed from a superelastic metal alloy, e.g., a nickel-titanium alloy. In embodiments, the splines 216A-216F each have lateral edges having an atraumatic shape. In some embodiments, an electrically insulative material is disposed on the support member 220, such as a parylene or poly ether block amide (PEBA) coating, such as an insulative material available under the trade designations PEBAX from Arkema S.A. of Colombes, France, or VESTAMID E from Evonik Industries AG of Essen, Germany. In some embodiments, the nickel-titanium alloy is insulated with a secondary material like a polymer such as a polyimide or similar material.

The support member 220 includes a support member hub 224 and a plurality of support member branches 226A-226F. In embodiments, the support member branches 226A-226F are integrally formed with and extend proximally from the support member hub 224. For example, the entire support member 200 may be cut from a single sheet of material using conventional manufacturing techniques. This unitary structure provides robust structural properties, for example, selective flexibility and enhanced fatigue characteristics, particularly in areas that are subject to relatively high stresses during manufacture and use of the electroporation catheter 200. Forming the support member 220 from a superelastic material such as a nickel-titanium alloy facilitates configuring the support member 220 to assume its desired unconstrained shape due to the shape memory properties of the material, while providing sufficient flexibility necessary to collapse the mapping electrode assembly 210 within a delivery sheath. In embodiments, the support member branches 226A-226F can be selectively configured along their lengths to tune the mechanical characteristics of the electrode assembly 210.

In embodiments that include splines 216A-216F with a flexible circuit 222, the flexible circuit 222 includes a flex circuit hub 230 and a plurality of flex circuit branches 234A-234F. In embodiments, the flex circuit hub 230 is disposed over and secured to the support member hub 224. In embodiments, the flex circuit branches 234A-234F are integrally formed with the flex circuit hub 230, and each of the flex circuit branches 234A-234F is disposed over and secured to a respective one of the support member branches 226A-226F. The flexible circuit 222 comprises a layered construction including one or more dielectric substrate layers, and conductive traces formed thereon. Similar to the support member 220, the unitary construction of the flexible circuit 222 enhances its structural properties, for example, by minimizing joints or other discontinuities at regions subject to relatively high stresses during use.

As shown, the splines 216A-216F include a plurality of spline-disposed mapping electrodes, or sensing electrodes 250. In the illustrated embodiment, each of the spline-disposed, or spline, sensing electrodes 250 is disposed within a periphery of splines 216A-216F. In some embodiments, the mapping electrodes 250 are arranged on the outer surfaces of the splines 216A-216F in a manner such that a plurality of mapping electrodes 250 are spaced-apart and outwardly facing on the catheter 220, such as opposite the inner space 212. In the illustrated example, each of the distal-most spline sensing electrodes 250 is disposed within a periphery of a respective one of the radial segments of the flexible circuit 222. In the illustrated embodiment, the mapping electrode assembly 210 further includes a hub sensing electrode 264 centrally located on the flex circuit hub 230. In some embodiments, the mapping electrodes are disposed on an electrically insulative material, such as the parylene or PEBA coating, disposed on a nitinol spline. In embodiments, the hub sensing electrode 264 allows tissue surface mapping to be conducted in a “forward” manner, eliminating the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250.

In some embodiments, the structural functionality of the support member 220 can be provided by a suitably designed flexible circuit 222. As such, although the mapping electrode assembly 210 is described in detail as including the support member 220 as a primary structural member of the spline 216A-216F, in other embodiments the support member 220 can be omitted in its entirety and the corresponding functionality can be provided by the flexible circuit 222.

Some configurations of electrode assemblies comprising support structures utilize both ablation electrodes and sensing electrodes on the same support structure. Some of such configurations can suffer performance issues, such as poor lesions, because of low current density on the ablation electrode designs. For example, electrode surface area can function to divide the current from pulsed waveforms, which results in decreased depth of ablation lesions. Further, ablation electrodes with edges disposed about a main surface have been discovered to generate undesired edge effects that result in increasing the frequency and intensity of ablation artifacts.

In the particular illustrated embodiment, the electroporation catheter 200 includes an ablation electrode assembly 260 extending distally from the distal end 209 of the outer shaft 202. As shown, the ablation electrode assembly 260 extends partially into the inner space 212. In embodiments, the ablation electrode assembly 260 includes a central post 258 extending from the outer shaft 202 and an ablation electrode 262 disposed on the central post 258. As further shown, in the particular illustrated embodiment, an optional irrigation lumen 261 is supported by the ablation electrode assembly 260 such as disposed on the central post 258. In embodiments, the ablation electrode assembly 260 may house additional components. For example, in embodiments, a magnetic navigation sensor (not shown) may be partially or wholly disposed within the central post 258. However, in other embodiments, a magnetic navigation sensor may be located elsewhere on the electroporation catheter 200 (e.g., within the outer shaft 202). In one example, the ablation electrode assembly 260 includes a diameter of approximately 8 French (Fr) (approximately 2.667 millimeters). The axial length of the ablation electrode assembly 260 can be varied, e.g., such as to extend into the proximal section of the inner space 212 or the distal section of the inner space 212, or variable by a user, such as retractable or extendable to various axial location, for instance, in the inner space 212. In embodiments, the electrodes 250, 260, 264 may have a coating of a suitable biocompatible metal, e.g., gold. In embodiments, the outer surfaces of the electrodes 250, 260, 264 may be treated to provide the electrical properties desired for the particular clinical application.

The illustrated embodiment of the ablation electrode assembly 260 includes the post ablation electrode 262 disposed on the distal end of the central post 258, although other configurations of an ablation electrode or a plurality of ablation electrodes on the central post 258 are contemplated. In the example, ablation electrodes of the catheter 200 in a monopolar mode are only carried on the central post 258. No ablation electrodes or electrodes configured for ablation in a monopolar mode are carried on the splines 216A-216F. In the illustrated embodiment, the ablation electrode 262 includes a circular planar exposed surface, with no exposed edges or longitudinally extending sides. The circular-surface ablation electrode 262 provides for surface area concentration and removes edge effects while concentrating current density to provide deep lesions. In another embodiment, the ablation electrode can be circular and domed, or with a curved surface having a distalmost point in the center of the and without exposed edges. Edges of the ablation electrode 260 can be covered in an insulative material. For instance, longitudinally extending surfaces of the electrode 262 are covered in insulation, which can extend distally to be flush with the flat surface of the circular planar electrode.

The post ablation electrode 262 can be configured with the electrophysiology system 50 (FIG. 1) to provide a number of functions in addition to ablation, such as when not configured to receive pulse field ablation waveform. In one example, the post ablation electrode 262 can operate as a reference for unipolar electrograms, in lieu of reliance on surface ECG patch electrodes as are otherwise known in the art. The location of the post ablation electrode 262 for this purpose positions the reference electrode much closer to the tissue being sensed than is possible with the conventional surface ECG approach, which may advantageously minimize far field noise and provide much sharper unipolar electrograms than what are possible using surface ECG electrodes. In another example, the ablation electrode assembly 260 can be configured to apply a radiofrequency (RF) energy via coupling the catheter 200 to an RF generator. The post ablation electrode 262 can be configured to apply RF energy and perform RF ablation such as in an example in which the post ablation electrode 262 is pressed into contact with target tissue.

The post ablation electrode 262 may also be operable to sense and measure other electrical parameters, e.g., voltages between it and other electrodes, such as other sensing electrodes 250 and the central hub electrode 264 on the electrode assembly 210, thereby providing data usable for, in some examples, determining the shape of the electrode assembly during use (including when deformed by forces applied by cardiac walls), and displaying shape information via the EAM system 70 (FIG. 1). For example, electrodes on the electrode assembly 210 can be used with reference voltages to determine whether the support structures such as splines 216A-216F have been deformed, such as the shape of the structures have deviated from expanded configuration, or deflected, such as whether the splines 216A-216F have been hinged off axis. For example, the stiffness of the splines 216A-216F in the expanded configuration can be tunes so as to deflect or deform under a force. Based on the determined shape of the mapping electrode assembly 210, or the relation of the electrodes 250, 262, 264 with respect to each other, the amount of force in the axial or radial directions can be determined, such as when the electrode assembly 210 is pressed against target tissue. Additionally, the amount of hinge of the electrode assembly 210 at the proximal portions of the splines 218A-218F can be determined. Further, the post ablation electrode 262 can detect its distance from target tissue or whether the post ablation electrode 262 is in contact with target tissue.

In the various embodiments, each of the electrodes, such as spline sensing electrodes 250, post ablation electrode 262, and the hub sensing electrode 264 are separately electrically connected to the control system of the electroporation console 130 (FIG. 1) and are individually addressable to provide for a wide range of ablation and sensing modes, e.g., monopolar and bipolar modes. During monopolar ablation operation, the post ablation electrode 262 is configured to operate at one polarity, and an electrode located elsewhere (e.g., a dispersive electrode located on the patient, typically on the back, buttocks, or other suitable anatomical location, or an electrode on a different catheter or probe located outside the cardiac chamber in which the electrode assembly 210 is located) is configured to operate at the opposite polarity. In one example, the post ablation electrode 262 is configured as an anode or cathode, and an extracorporeal dispersive electrode located on a back patch is configured as the other of the cathode or anode.

During bipolar ablation operation, a first set of one or more ablation electrodes of the electrode assembly 210 is configured as the anode (or cathode) and a second set of one or more other ablation electrodes of the electrode assembly 210 is configured as the cathode (or anode). In examples, the bipolar ablation electrode sets can comprise electrodes on different splines or can be formed between one or more of the sensing electrodes 250 and the post ablation electrode 260. In another example, the bipolar set can comprise the post ablation electrode 260 and the hub sensing electrode 264. In another example, the bipolar set can comprise the post ablation electrode 260 and the shaft electrodes 256. Similarly, any of the spline sensing electrodes 250, the post ablation electrode 260 or the hub sensing electrode 264 can also be individually addressed for bipolar sensing and mapping an any number of combinations.

FIGS. 2B-2D are partial perspective illustrations of electroporation catheters 200b-200d that can be configured for use as catheter 105 and include alternative embodiments of mapping electrode assembly 210 and ablation electrode assembly 260 of catheter 200. In each of the illustrated embodiments, the electroporation catheters 200a-200d include a tubular outer shaft 202b-200d having a shaft distal end 209b-200d, a mapping electrode assembly 210b-210d extending distally from the respective distal end 209b-209d and defining an inner space 212, and an ablation electrode assembly 260b-260d extending distally from the respective distal ends 209b-209d and disposed in the inner space 212. Mapping electrode assembly 210b-210d includes sensing electrodes 250b-250d, respectively. FIG. 2B illustrates an ablation electrode assembly 260b having an ablation electrode 262b disposed at a distal end of a central post 258b, the central post 258b extending distally from the shaft distal end 209b into the inner space 212. In the illustrated example, the ablation electrode assembly 260b extends into the distal portion of the inner space 212, such as proximate the support member hub 224b of the mapping electrode assembly 260b. FIG. 2C illustrates an ablation electrode assembly 260c having a plurality of ablation electrodes 262c-1, 262c-2 disposed on a central post 258c, the central post 258c extending distally from the shaft distal end 209c into the inner space 212. In the illustrated example, the ablation electrode assembly 260c extends into the medial portion of the inner space 212. FIG. 2D illustrates an ablation electrode assembly 260d having an ablation electrode 262d disposed at a distal end of a central post 258d, the central post 258d extending distally from the shaft distal end 209d into the inner space 212. In the illustrated example, the ablation electrode 262d includes a diameter larger than a diameter of the central post 258d.

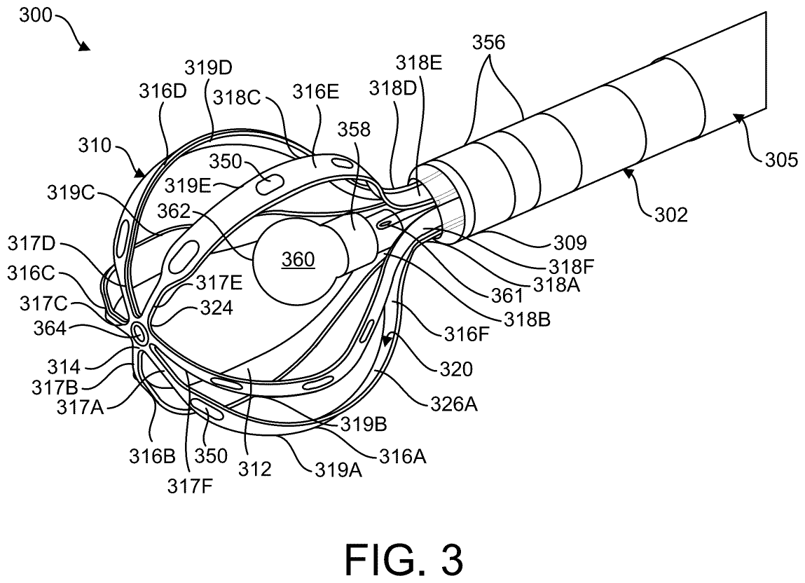

FIG. 3 is a partial perspective illustration of another embodiment of an electroporation catheter 300 and can also correspond to the electroporation catheter 100 described with respect to FIG. 1. Features of the embodiment of electroporation catheter may be include on the embodiment of electroporation catheter 200 (FIG. 2), and vice versa. The electroporation catheter 300 includes a catheter distal portion 305 according to an embodiment of the present disclosure. The electroporation catheter 300 has a tubular outer shaft 302 having a shaft distal end 309, a mapping electrode assembly 310 extending distally from the distal end 309 of the outer shaft 302, and an ablation electrode assembly 360. In embodiments, the electrode assembly 310 is configured to self-expand from a collapsed configuration when constrained within a delivery sheath to a pre-defined expanded configuration defining an inner space 312. The ablation electrode assembly 360 extends distally from the distal end 309 of the outer shaft 202 and is disposed in the inner space 312. The ablation electrode assembly 360 is configured to receive pulsed electrical signals/waveforms from the electroporation console 130 (FIG. 1), thereby creating pulsed electric fields sufficient for ablating target tissue via irreversible electroporation. The mapping electrode assembly 310 includes a plurality of mapping and sensing electrodes 350 configured for sensing cardiac electrical signals, localization of the electrode assembly 310 within the patient anatomy (e.g., via the EAM system 70 of FIG. 1), and determining proximity to target tissue within the anatomy.

The mapping electrode assembly 310 has a distally-located central hub portion 314 and a plurality of splines 316A-316F extending proximally from the central hub portion 314. As further shown, each respective spline 316A-316F has a distal end portion 317A-317F, a proximal end portion 318A-318F, and an intermediate portion 319A-319F extending between the distal end portion 317A-317F and the proximal end portion 318A-318F. The particular geometry of the splines 316A-316F and the related components, e.g., mapping electrodes 350, is optimized to provide desired mechanical and therapeutic/diagnostic capabilities.

In one embodiment, the splines 316A-316F are composed of a support member 320 and a flexible circuit 322 secured to and disposed over an outer surface of the support member 322. The support member 320 functions, among other things, as a primary structural support of the mapping electrode assembly 310, and thus primarily defines the mechanical characteristics of the mapping electrode assembly 310. In embodiments, the support member 320 is formed from a superelastic material (metal or polymer) to provide desired mechanical/structural properties to the electrode assembly 310. In embodiments, the support member 320 is formed from a superelastic metal alloy, e.g., a nickel-titanium alloy. Forming the support member 320 from a superelastic material such as a nickel-titanium alloy facilitates configuring the support member 320 to assume its desired unconstrained shape due to the shape memory properties of the material, while providing sufficient flexibility necessary to collapse the electrode assembly 310 within a delivery sheath. In embodiments, the support member branches 326A-326F can be selectively configured along their lengths to tune the mechanical characteristics of the electrode assembly 310. In some embodiments, the structural functionality of the support member 320 can be provided by a suitably designed flexible circuit 322. As such, although the electrode assembly 310 is described in detail as including the support member 320 as a primary structural member, in other embodiments the support member 320 can be omitted in its entirety and the corresponding functionality can be provided by the flexible circuit 322. In still other embodiments, the flexible circuit can be omitted entirely, and the mapping electrodes 350 are suitably attached to the support member 320 of the splines 316A-316F.

As shown, the flexible circuit 322 includes a plurality of spline-disposed mapping electrodes, or sensing electrodes 350. In the illustrated embodiment, each of the spline-disposed, or spline, sensing electrodes 350 is disposed within a periphery of the spline, such as within the periphery of the flexible circuit 322. For example, as shown, each of the distal-most spline sensing electrodes 350 is disposed within a periphery of a respective one of the radial segments of the flexible circuit 322.

In the illustrated embodiment, a spline 316A, a subset of the plurality of splines 316A-316n, or all of the plurality of spline 316A-316F, are angularly movable with respect to the other spines. For example, one spline 316F may be rotated about the longitudinal axis to approach another spline or to move away from another spline, such as spline 316E, so that the plurality of splines are not evenly spaced apart from each other angularly about the longitudinal axis as illustrated. The splines 316A-316F can be pivotally connected to the central hub portion 314 or not connected to the central hub portion 314. this enables the main ablation electrode 360 to be exposed from behind the splines 316A-316F.

The illustrated embodiment includes an ablation electrode assembly 260 having a central post 358 extending distally from the distal end 309 of the outer shaft 302. As shown, the central post 358 extends partially into the inner space 312 and includes a post ablation electrode 360. As further shown, in the particular illustrated embodiment, the ablation electrode assembly includes an optional irrigation lumen 361 is supported by the central post 358.

The post ablation electrode 360 in the illustrated embodiment is disposed on the distal end of the central post 358, although other configurations of an ablation electrode or a plurality of ablation electrodes on the central post 358 are contemplated. In the example, ablation electrodes of the catheter 300 in a monopolar mode are only carried on the central post 358. No ablation electrodes or electrodes configured for ablation in a monopolar mode are carried on the splines 316A-316F. In the illustrated embodiment, the ablation electrode 360 includes a bulbous three-dimensional object having a conductive exposed surface, with no exposed edges or longitudinally extending sides. The bulbous ablation electrode 360 provides for surface area concentration and removes edge effects while concentrating current density to provide deep lesions. Edges of the ablation electrode 360 can be covered in an insulative material. For instance, longitudinally extending surfaces of the electrode 360 are covered in insulation, which can extend distally to be flush with the flat surface of the circular planar electrode.

The shaft distal end 309 can carry shaft electrodes 356 to configured the catheter 300 in a bipolar mode.

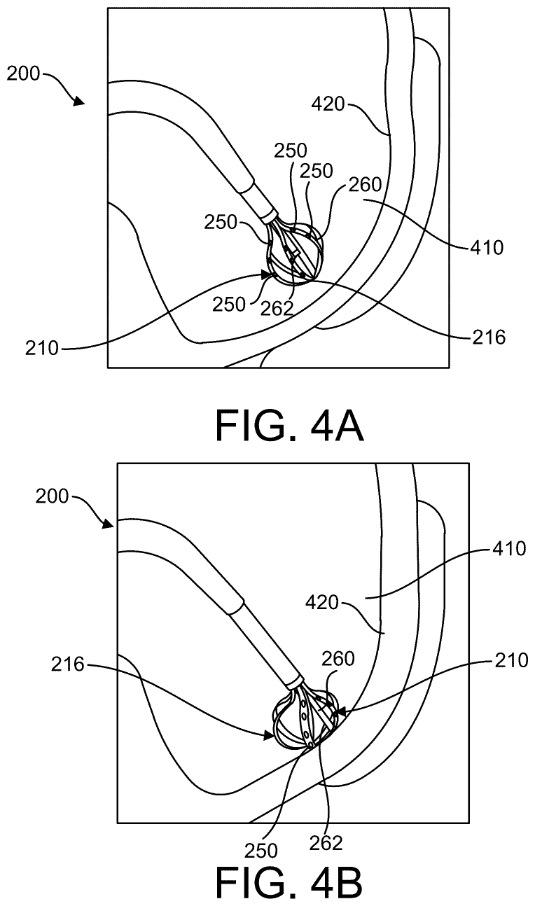

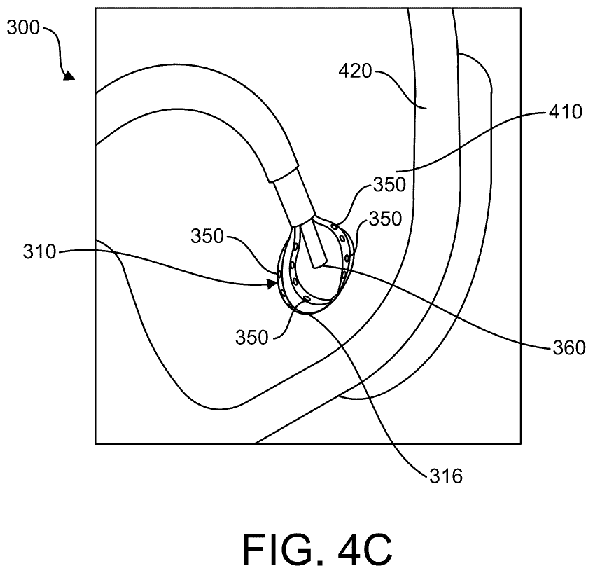

FIGS. 4A and 4B illustrate the functionality of catheters 200, 300, and FIG. 4C illustrates another functionality of catheter 300. In particular, FIGS. 4A-4C illustrate the functionalities of the mapping electrode assemblies 210, 310 in use in a cardiac procedure. In the illustrated examples, the electroporation catheter 200 includes a tubular outer shaft 202 having a shaft distal end 209, a mapping electrode assembly 210 extending distally from the respective distal end 209 and defining an inner space 212, and an ablation electrode assembly 260 extending distally from the respective distal ends 209 and disposed within the inner space 212. As illustrated in FIG. 4A, the mapping electrode assembly 210 can be positioned within a cardiac chamber of interest, e.g., the left atrium 410. When positioned within the blood pool of the left atrium 410, the mapping electrode assembly 210 is pre-configured to assume its fully expanded shape. As shown in FIG. 4B, the design of the mapping electrode assembly 210 allows the splines 216 to elastically deform when the electrode assembly 210 is urged into contact with the wall 420 of the cardiac chamber. The splines may be deformed by axial forces from the tip, radial forces on the splines, or hinged forces as illustrated in FIG. 4B. The illustrated deformation results in maximizing the surface area of the and sensing electrodes 250 that are in contact with the target tissue without placing undesirable force on the cardiac wall 420. In one example, the mapping electrode assembly 210 is deformable with approximately a 50-gram force of contact with the wall 420 of the cardiac chamber to allow the ablation electrode assembly 260 or in particular the ablation electrode, to contact the wall 420. The stiffness of the ablation electrode assembly 260 is tuned to provide a larger footprint of ablation once the mapping electrode assembly 210 is deformed (such as axially deformed, radially deformed, or hinged if the mapping electrode 210 is coupled to the shaft 202 via a hinge mechanism) such as being pressed against the wall. The flexibility of the mapping electrode assembly 210 further results in its assumption of its undeformed expanded shape when it is retracted away from the cardiac wall 420. FIG. 4C illustrated electrode assembly 310 when splines are moved aside to allow expose the main ablation electrode 360 within the inner surface to the cardiac wall 420.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for ablating cardiac tissue through irreversible electroporation, the catheter comprising:
a tubular outer shaft having a distal end;
a mapping electrode assembly extending distally from the distal end of the outer shaft, the mapping electrode assembly defining a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes; and
an ablation electrode assembly extending distally from the distal end of the outer shaft, the ablation electrode assembly disposed in the inner space.

2. The catheter of claim 1, wherein the ablation electrode assembly is movable with relation to the mapping electrode assembly in the expanded configuration.

3. The catheter of claim 1, further comprising a hub sensing electrode centrally located on the central hub portion of the electrode assembly.

4. The catheter of claim 1, wherein the splines each have lateral edges having an atraumatic shape.

5. The catheter of claim 1, wherein the ablation electrode assembly includes a central post and an ablation electrode, the central post extending distally from the distal end of the tubular shaft and into the inner space defined by the mapping electrode assembly when the mapping electrode assembly is in an expanded configuration, wherein the ablation electrode is disposed on the central post.

6. The catheter of claim 1, wherein the ablation electrode assembly includes a circular surface of an exposed conductive material generally perpendicular to a longitudinal axis of the catheter.

7. The catheter of claim 1, wherein the ablation electrode assembly includes a domed shape of an exposed conductive material.

8. The catheter of claim 1, wherein the ablation electrode assembly includes a bulbous shape of an exposed conductive material.

9. The catheter of claim 1, wherein the ablation electrode assembly includes a plurality of ablation electrodes.

10. The catheter of claim 1, wherein a movable spine of the plurality of splines is angularly movable with respect to another spline of the plurality of splines.

11. The catheter of claim 1, wherein each spline of the plurality of splines are fixed in relation to all other splines of the plurality of splines.

12. The catheter of claim 1, wherein the plurality of splines includes a support member.

13. The catheter of claim 1, wherein each of the splines includes a flex circuit comprising the spline sensing electrodes.

14. The catheter of claim 13, wherein each of the splines further includes a support member having an outer surface and the flex circuit comprising the spline sensing electrode is disposed on the outer surface of the support member.

15. The catheter of claim 13, wherein the support member comprises a nickel-titanium alloy.

16. A catheter for ablating cardiac tissue through irreversible electroporation, the catheter comprising:
a tubular outer shaft having a distal end;
a mapping electrode assembly extending distally from the distal end of the outer shaft, the electrode assembly defining a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes disposed on an outer surface of a support member; and
an ablation electrode assembly extending distally from the distal end of the outer shaft, wherein the ablation electrode assembly includes a central post and an ablation electrode, the central post extending distally from the distal end of the tubular shaft and into the inner space defined by the mapping electrode assembly when the mapping electrode assembly is in an expanded configuration, wherein the ablation electrode is disposed on the central post.

17. The catheter of claim 16, wherein each of the splines includes a flex circuit comprising the spline sensing electrodes.

18. The catheter of claim 16, wherein the ablation electrode includes an exposed conductive material generally perpendicular to a longitudinal axis of the catheter.

19. A catheter for ablating cardiac tissue through irreversible electroporation, the catheter comprising:
a tubular outer shaft having a distal end;
a mapping electrode assembly extending distally from the distal end of the outer shaft, the mapping electrode assembly defining a distally located central hub portion and a plurality of splines each including a distal end portion extending from the central hub portion, and a proximal end portion attached to and constrained by the outer shaft, the splines defining an inner space in an expanded configuration, each of the plurality of splines including a plurality of outwardly-facing sensing electrodes, wherein a movable spine of the plurality of splines is angularly movable with respect to another spline of the plurality of splines; and
an ablation electrode assembly extending distally from the distal end of the outer shaft, the ablation electrode assembly disposed in the inner space, wherein the ablation electrode assembly is movable with relation to the mapping electrode assembly in the expanded configuration.

20. The catheter of claim 19, wherein the ablation electrode assembly includes a central post and an ablation electrode, the central post extending distally from the distal end of the tubular shaft and into the inner space defined by the mapping electrode assembly when the mapping electrode assembly is in an expanded configuration, wherein the ablation electrode is disposed on the central post.
